(19) United States
(12) Patent Application Publication
(10) Pub. No.: US 2015/0299200 A1
(43) Pub. Date: Oct. 22, 2015

(54) AN IMPROVED PROCESS FOR THE PREPARATION OF LINAGLIPTIN

(71) Applicants: Bhausaheb CHAVHAN, (US); Jebaraj RATHINAPANDIAN, (US); Shiva Kumar CHANDUPTLA, (US); Nagaraji GHANDA, (US); MYLAN LABORATORIES LTD, Hyderabad (IN)

(72) Inventors: Bhausaheb CHAVHAN, Hyderabad (IN); Jebaraj RATHINAPANDIAN, Hyderabad (IN); Shiva Kumar CHANDUPTLA, Hyderabad (IN); Nagaraji GHANDA, Hyderabad (IN)

(73) Assignee: MYLAN LABORATORIES LTD, Hyderabad (IN)

(21) Appl. No.: 14/652,230

(22) PCT Filed: Dec. 16, 2013
(86) PCT No.: PCT/IN2013/000773

§ 371 Date: Jun. 15, 2015

(30) Foreign Application Priority Data
Dec. 17, 2012 (IN) 5242/CHE/2012

(51) Int. Cl. C07D 473/06

(52) U.S. Cl. CPC C07D 473/06

(57) ABSTRACT

The present invention relates to a process for preparing Linagliptin by purifying the intermediate compounds converting the purified intermediates into Linagliptin. The present invention also relates to the preparation of an amorphous Linagliptin.

This application claims priority to this Indian patent application number 5242/CHE/2012 dated on Dec. 17, 2012.

### FIELD OF INVENTION

The present invention relates to an improved process for the preparation of Linagliptin and further relates to process for the preparation of amorphous Linagliptin.

### BACKGROUND OF THE INVENTION

Linagliptin is chemically described as 8-[(3R)-3-aminopiperidin-1-yl]-7-(but-2-yn-1-yl)-3-methyl-1-[(4-methylquinazolin-2-yl)methyl]-3,7-dihydro-1H-purine-2,6-dione having the structural formula I.

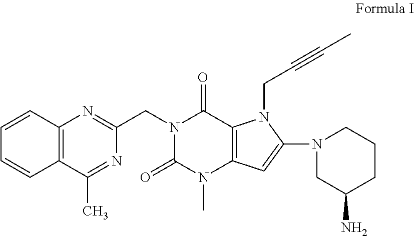 [SMILES: CC#CCn1c(N2CCC[C@@H](N)C2)cc2c1c(=O)n(Cc1nc(C)c3ccccc3n1)c(=O)n2C]

Linagliptin acts as an active inhibitor of the dipeptidyl peptidase-4 (DPP-4) enzyme, developed by Boehringer Ingelheim and marketed under the brand name of TRADJENTA.

U.S. Pat. No. 7,407,955 discloses a group of inhibitors of the dipeptidyl peptidase-4 (DPP-4) enzyme and claims Linagliptin and its physiologically acceptable salts. Journal of Medicinal Chemistry 2007, 50, 6450-6453, discloses process for the preparation of Linagliptin.

US 2012129874 describes crystalline acid addition salts of Linaglitpin. US 20070259900 described crystalline form A, B, C, D and E of Linagliptin. The amorphous form of Linagliptin is disclosed in IP.com Journal (2011), 11(9A), 22.

### OBJECT AND SUMMARY OF THE INVENTION

The principle object of the present invention is to provide Linagliptin with improved yield and purity.

Yet another object of the present invention is to provide a process for the preparation of an amorphous form of Linagliptin.

In one aspect, the present invention provides an improved process for preparing Linagliptin comprising the steps of:
a) brominating the methyl xanthine compound of formula A to give a bromo xanthine compound of formula B,
b) reacting the bromo xanthine compound of formula B with a butynyl compound of formula H to give a butyl xanthine compound of formula C,
c) condensing the butyl xanthine compound of formula C with a quinazoline compound of formula D to give a compound formula E,
d) reacting the compound of formula E with a protected piperidine compound of formula F to give a protected Linagliptin compound of formula G,
e) deprotecting the protected compound of formula G to give Linagliptin compound of formula I.

In one more aspect, the present invention provides a process for preparing amorphous form of Linagliptin comprising the steps of:
a) dissolving Lingaliptin in a suitable solvent, and
b) removing the solvent to isolate amorphous Linagliptin.

### BRIEF DESCRIPTION OF THE DRAWINGS

FIG. 1: A powder X-ray diffraction pattern of compound of Formula G.

### DETAILED DESCRIPTION OF INVENTION

The present invention provides an improved process for the preparation of Linagliptin with improved yield and purity. It also provides a process for the preparation of an amorphous form of Linagliptin.

### Instrumentation

### Powder X-ray Diffraction (PXRD)

The polymorph of the present invention is characterized by their X-ray powder diffraction patterns. Thus, X-ray diffraction patterns of the polymorph of the invention were measured on BRUKER D-8Discover powder diffractometer equipped with (a?) goniometer of θ/2θ configuration and Lynx Eye detector. The Cu-anode X-ray tube was operated at 40 kV and 30 mA. The experiments were conducted over the 20 range of 2.0°-50.0°, 0.030° step size and 0.4 seconds step time.

The schematic representation of the present invention is shown in scheme 1:

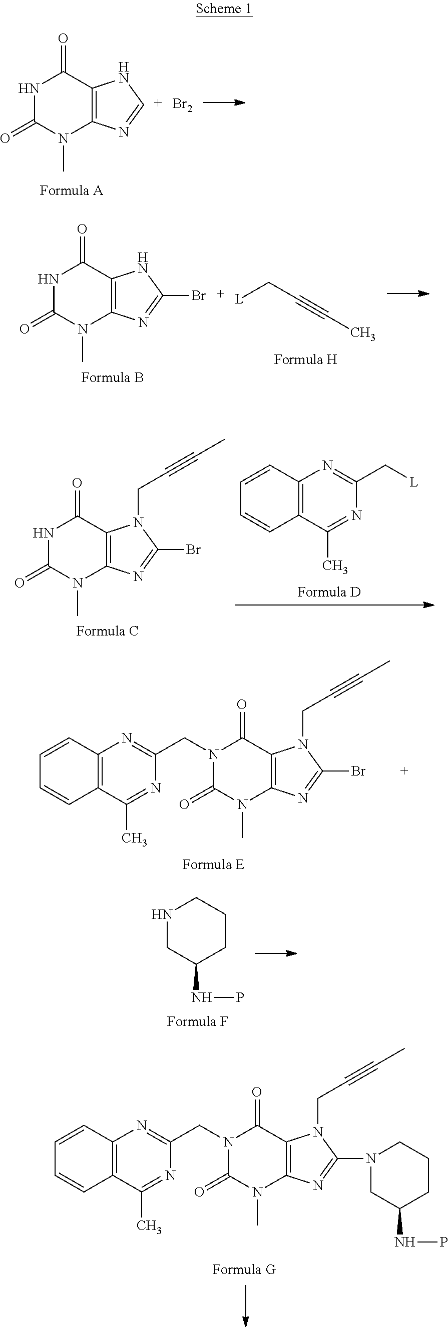

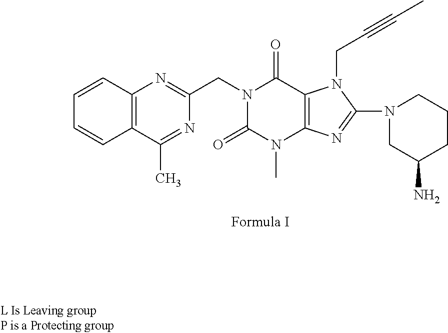 [SMILES: CC#CCn1c(N2CCC[C@@H](N)C2)nc2c1c(=O)n(Cc1nc(C)c3ccccc3n1)c(=O)n2C]

As used herein, “L” refers to a leaving groups known in the art includes, halides, sulphonate groups such as alkylsulphonates arylsulphonates.

“P” refers to a protecting group known in the art includes di t-Butyl dicarbamate (Boc), benzyl carbamate, acetamide, trifluoroacetamide, phthalimide, benzylchloride, benzoylchloride, tritylamine, benzylideneamine, tosylamide, methoxyethxoxymethyl, tetrahydropyaranyl (THP), and tertiary butyl.

Halide refers to, F, Cl, Br, and I.

In one aspect, the present invention provides an improved process for the preparation of Linagliptin comprising the steps of:
a) brominating the methyl xanthine compound of formula A to give a bromo xanthine compound formula B,
b) reacting the bromo xanthine compound of formula B with a butynyl compound of formula H to give a butyl xanthine compound of formula C,
c) condensing the butyl xanthine compound of formula C with a quinazoline compound of formula D to give a compound formula E,
d) reacting the compound of formula E with a protected piperidine compound of formula F to give a protected Linagliptin compound of formula G,
e) deprotecting the protected compound of formula G to give Linagliptin compound of formula I.

In one embodiment of the present invention, the methyl xanthine compound of formula A is brominated by reacting compound of formula A with liquid bromine in the presence of a metal acetate and a suitable solvent such as acetic acid to give a bromo xanthine compound of formula B. The metal acetate includes sodium acetate, potassium acetate, preferably sodium acetate. The resulting intermediate compound of formula B is purified by treating with an organic solvent such as methanol, ethanol, propanol, isopropanol, butanol, preferably methanol to give pure intermediate compound of formula B.

U.S. Pat. No. 7,407,955 process employs potassium carbonate and acetonitrile for the bromination of the xanthine compound of formula A in which, the reaction doesn't comply totally and results into less yield and purity. As per the present invention, by employing bromination reaction in presence of sodium acetate gives more purity and yield.

In another embodiment of the present invention, the bromo xanthine compound of formula B is reacted with a butyne derivative of formula H in presence of a base and a suitable solvent to give compound of formula C. The suitable base is selected from diisopropyl ethyl amine, dimethylamine, triethyl amine (TEA), trimethyl amine, methylamine, ethanolamine, triphenylamine, pyridine and piperidine, preferably diisopropyl ethyl amine. The suitable solvent is selected from dimethyl formamide (DMF), dimethyl sulfoxide (DMSO), dimethylacetamide, tetrahydrofuran (THF), acetone, and acetonitrile preferably DMF. The resulting intermediate compound of formula C is purified by treating with an organic solvent such as methanol, ethanol, propanol, isopropanol, butanol, preferably methanol to give pure Intermediate compound of formula C.

In one more embodiment of the present invention, the butyl xanthine compound of formula C is condensed with a quinazoline compound of formula D in presence of a base and suitable solvent and optionally in presence of a phase transfer catalyst to give compound of formula E. The suitable base is selected from metal hydroxide includes potassium hydroxide (KOH), sodium hydroxide (NaOH), metal carbonate includes sodium carbonate and potassium carbonate, preferably potassium carbonate. The suitable solvent is selected from polar aprotic solvent such as dimethyl sulfoxide (DMSO), dimethylacetamide, tetrahydrofuran (THF), acetone, dimethyl formamide (DMF) and acetonitrile; preferably DMSO. The phase transfer catalyst includes tetrabutyl ammonium bromide, tetrabutylammonium fluoride, tetrabutylammonium hydroxide, triethylmethylammonium bromide, benzyl tributylammonium bromide, hexadecylpyridinium bromide. The resulting intermediate compound of formula E is purified by treating with an organic solvent such as methanol, ethanol, propanol, isopropanol, butanol, preferably methanol to give pure Intermediate compound of formula E.

Yet another embodiment of the present invention, the compound of formula E is reacted with a protected piperidine compound of formula F, preferably P is Boc group, in presence of a base in a suitable solvent and optionally in presence of a metal halide to give compound of formula G. The suitable base is selected from metal hydroxide includes potassium hydroxide (KOH), sodium hydroxide (NaOH), metal carbonate includes sodium carbonate and potassium carbonate, preferably metal carbonate potassium carbonate. The suitable solvent is selected from polar aprotic solvent such as dimethyl sulfoxide (DMSO), dimethylacetamide, tetrahydrofuran (THF), acetone, dimethyl formamide (DMF) and acetonitrile; preferably DMSO. The metal halide includes potassium Iodide (KI). The resulting intermediate compound of formula G is purified by treating with an organic solvent such as dichloromethane, methanol, ethanol, propanol, isopropanol, butanol, preferably dichloromethane and hexanes, n-hexane or cyclohexane to give pure intermediate compound of formula G.

In another embodiment, to enhance the purity of the target compound Linagliptin, the intermediates of each stage of the present invention are purified with methanol.

In one embodiment of the present invention, the protected Linagliptin compound of formula G is purified through charcolization before proceeding to the deprotection, which results the Linagliptin with high purity. As per the present invention, the protected Linagliptin compound of formula G is dissolved in a suitable solvent such as dichloromethane, to this charcoal is added and stirred. The resultant reaction mixture is filtered and further converted into Linagliptin.

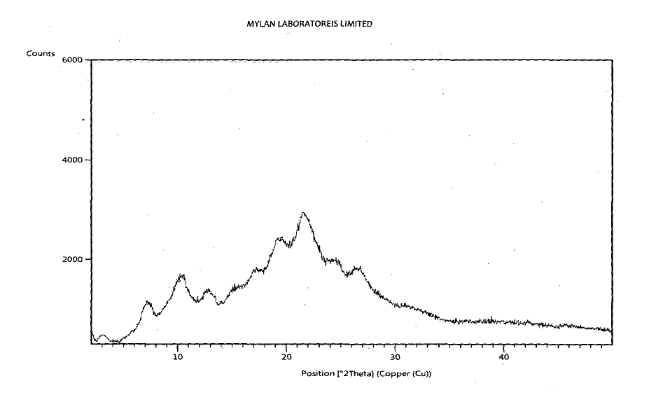

In an additional embodiment of the present invention, the purified compound formula G (wherein P is tert.butoxycarbonyl) obtained above after charcolization is characterized by a Powder X-ray diffraction pattern as depicted in FIG. 1, and is further converted into Linagliptin.

The process exemplified in U.S. Pat. No. 7,407,955 as shown in scheme 2 gives ˜80% of HPLC purity.

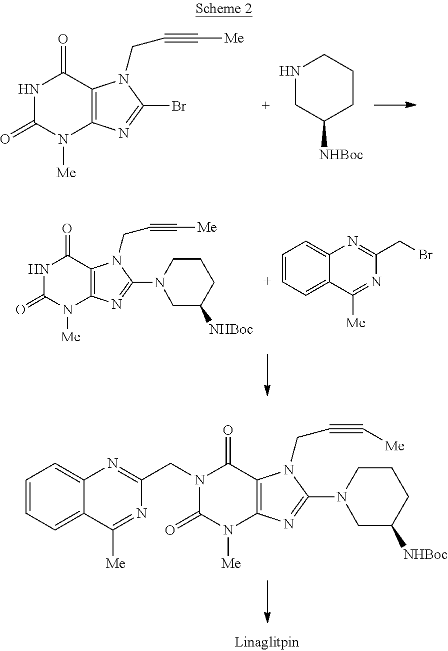 [SMILES: CC#CCn1c(Br)nc2c1c(=O)[nH]c(=O)n2C ; CC(C)(C)OC(=O)N[C@@H]1CCCNC1 ; CC#CCn1c(N2CCC[C@@H](NC(=O)OC(C)(C)C)C2)nc2c1c(=O)[nH]c(=O)n2C ; Cc1nc(CBr)nc2c1=C[C+]=CC=2 ; CC#CCn1c(N2CCC[C@@H](NC(=O)OC(C)(C)C)C2)nc2c1c(=O)n(Cc1nc(C)c3ccccc3n1)c(=O)n2C]

According to the present invention, more than 97% of HPLC purity of Linagliptin is obtained.

One more embodiment of the present invention, the compound of formula G is deprotected by treating with an acid in a suitable solvent to give Linagliptin of formula I. The suitable acid for the deprotection includes trifluoro acetic acid, hydrochloric acid, chlorotrimethylsilane preferably trifluoroacetic acid. The suitable solvent is selected from polar aprotic solvent such as dichloromethane, methanol, ethanol, isopropyl alcohol, ethyl acetate, acetone, dioxane, diethyl ether, carbon tetrachloride, and toluene; preferably dichloromethane.

U.S. Pat. No. 7,407,955 process employs isopropyl alcohol/hydrochloric acid for deprotection of formula G, which leads to the formation of more impurities, by using trifluoroacetic acid/methylene dichloride as described in the present invention, the formation of impurities level is less.

In one more aspect, the present invention provides a process for the preparation of an amorphous form of Linagliptin comprising the steps of:
c) dissolving Lingaliptin in a suitable solvent, and
d) removing the solvent to isolate an amorphous Linagliptin.

In one embodiment of the present invention, Linagliptin is dissolved in a suitable solvent selected from methanol, ethanol, 1,4-dioxane, tetrahydrofuran, methylene dichloride; preferably methylene dichloride and methanol.

In one more embodiment of the present invention, the solvent is removed by using methods selected from spray drying, freeze drying, agitated thin film dryer (ATFD) and distillation, preferably spray drying or distillation at 25-70° C., to isolate an amorphous Linagliptin.

The following non-limiting examples illustrate specific embodiments of the present invention. The examples are not intended to be limiting the scope of present invention in any way.

### EXAMPLES

### Example

### 1 Preparation of 8-bromo3-Methyl-xanthine

400 ml of Acetic acid, 100 g of 3-Methyl-xanthine(0.6019 moles) and 74 g of sodium acetate (0.9028 moles) were charged into a one lit. Round bottom flask fitted with overhead stirrer, thermo pocket and dropping funnel at 25-30° C. The mixture was stirred for 5-10 minutes and cooled to 10-15° C. To the reaction mixture was slowly added 144.2 g of liquid bromine (0.9028 moles) drop wise for about 60 minutes and the temperature was raised to 60-65° C.; and maintained for 3-4 hrs. After completion of the reaction, the reaction mixture was cooled to 15-20° C. and was slowly added 800 ml of DM water; The reaction mixture was maintained under stirring for 2-3 hrs. The obtained solid was filtered and washed with DM water. DM water slurry wash was given to the wet material and the wet material was charged into RB flask. To the wet material was added 700 ml of methanol and the temperature was raised to 60-65° C.; and maintained for 60 min at 60-65° C. The reaction mixture was cooled to 40-45° C. and maintained for 60 minutes. The resulting solid was filtered and washed with methanol. The wet material was dried under vacuum at 40-45° C. for 5-8 hours to get title compound (125-135 g, 92%, purity >99.5%).

### Example: 2

### Preparation of 3-methyl-7-(2-butyn-1-yl)-8-bromo-xanthine

1000 ml of DMF, 62 g of N, N-Diisopropyl ethylamine (0.6128 moles) and 100 g of 8-bromo-3-Methyl-xanthine (0.4081 moles, prepared as per Example-1) were added to a 5 lit. R. B. flask equipped with overhead stirrer, thermo pocket and dropping funnel at 20-30° C. and stirred for 5-10 minutes to get clear solution. To the reaction mixture was slowly added 81.45 g of 1-Bromo-2-butyne (0.6128 moles) at 25-30° C. and maintained the reaction mixture at same temperature for 3-4 hrs. After completion of the reaction, slowly added 2000 ml of chilled DM water to the reaction mixture and stirred for 1-2 hrs at 25-30° C. The solid was filtered and washed with 100 ml of DM water. The wet material was charged into RB flask and charged 700 ml of methanol and the temperature was raised to 60-65° C. and maintained for 60 min. The reaction mixture was cooled to 40-45° C. and maintained for 60 minutes. The solid was filtered and washed with 100 ml of methanol; Dried at 40-45° C. for 5-8 hours to get title compound (106 g, 87.6%, purity >99%).

### Example: 3

### Preparation of 1-[(4-methyl-quinazolin-2yl) methyl]-3-methyl-7-(2-butyn-1-yl)-8-Bromo-xanthine

700 ml of DMSO, 77.8 g of 2-(Chloromethyl)-4-methyl-quinazoline (0.4038 moles), 100 g of 3-methyl-7-(2-butyn-1-yl)-8-bromo-xanthine (0.3365 moles, prepared in Example-2), 0.5 g tetrabutyl ammonium bromide and 55.8 g of anhydrous potassium carbonate (0.4038 moles) were added into a 5 lit a round bottom flask equipped with overhead stirrer & thermo pocket at 20-30° C. and the temperature was raised to 75-80° C. The reaction mixture was maintained at 75-80° C. for 2-3 hrs. After completion of the reaction, reaction mixture was cooled to 45-50° C. To the reaction mixture was slowly added 600 ml of methanol and stirred for 60 min at 45-50° C. The solid was filtered and washed with 200 ml of methanol followed by DM water slurry. The wet material was charged into RB flask and charged 700 ml of methanol into RB flask; the temperature was raised to 65° C. and maintained for 60 min. The reaction mass was cooled to 40-45° C. and maintained for 60 minutes. Filtered the solid and washed with 200 ml methanol. The wet material was dried at 40-45° C. for 5-8 hours to get title compound (128 g, yield −84%, purity >99%).

### Example 4

### Preparation of 1-[(4-methyl-quinazolin-2yl) methyl]-3-methyl-7-(2-butyn-1-yl)-8-[(R)-3-(tert.-butyloxycarbonylamino)-piperidin-1-yl]-xanthine

800 ml of DMSO, 53.2 g of (R) 3-Boc-Aminopiperidine (0.2654 moles), 100 g of 1-[(4-methyl-quinazolin-2yl)methyl]-3-methyl-7-(2-butyn-1-yl)-8-Bromo-xanthine (0.2212 moles prepared as per Example-3), 0.5 g of potassium iodide and 91.5 g of potassium carbonate (0.6620 moles) were charged into a 5 lit round bottom flask equipped with overhead stirrer and thermo pocket at 20-30° C. The reaction mixture temperature was raised to 80-85° C. and maintained for 4-5 hrs at same temperature. After completion of the reaction, reaction mixture was cooled to 30-35° C., slowly added 1600 ml of chilled DM water and stirred for 60 min at 25-35° C. The solid was filtered and washed with 200 ml of DM water. The wet material again washed with DM water. The wet material charged into RB flask and charged 700 ml of dichloromethane and stirred for 30 mins and layers were separated. The organic layer was washed with DM water and treated with activated charcoal followed by filtration through hyflo and washing with dichloromethane. The solvent was distilled out U/V at 35-40° C. till ˜1.5V Dichloromethane remained inside. In another RB flask charged 800 ml of hexanes/cyclohexane and above dichloromethane solution was added slowly 35-40° C. and stirred for 30-60 minutes at 30-35° C. The solid was filtered and washed with 200 ml of hexanes/cyclohexane. The wet material was dried under vacuum at 40-45° C. for 5-8 hours to get title compound (115 g, 91%, purity >97%).

### Example 5

### Preparation of 1-[(4-methyl-quinazolin-2yl) methyl]-3-methyl-7-(2-butyn-1-yl)-8-[(R)-3-(tert.-butyloxycarbonylamino)-piperidin-1-yl]-xanthine

800 ml of DMSO, 53.2 g of (R) 3-Boc-Aminopiperidine (0.2654 moles), 100 g of 1-[(4-methyl-quinazolin-2yl)methyl]-3-methyl-7-(2-butyn-1-yl)-8-Bromo-xanthine (0.2212 moles prepared as per Example-3), 45.8 g of potassium carbonate (0.3318 moles) were charged into a 5 lit round bottom flask equipped with overhead stirrer and thermo pocket at 20-30° C. The reaction mixture temperature was raised to 80-85° C. and maintained for 4-5 hrs at same temperature. After completion of the reaction, reaction mixture was cooled to 30-35° C., and was slowly added 1600 ml of chilled DM water and stirred for 60 min at 25-35° C. The resulting solid was filtered and washed with 200 ml of DM water. The wet material was dried under vacuum at 50-65° C. for hours to get title compound (125 g).

### Example 6

### Preparation of 1-[(4-methyl-quinazolin-2yl) methyl]-3-methyl-7-(2-butyn-1-yl)-8-[3-(R) amino-piperidin-1-yl]-xanthine (Linagliptin)

500 ml of Dichloromethane and 100 g of 1-[(4-methyl-quinazolin-2yl) methyl]-3-methyl-7-(2-butyn-1-yl)-8-[(R)-3-(tert-butyloxycarbonylamino)-piperidin-1-yl]-xanthine (0.1746 moles prepared in example-4) were added into 1 lit round bottom flask equipped with overhead stirrer, thermo pocket and dropping funnel at 25-30° C. To the reaction mixture was slowly added 200 ml of trifluoroacetic acid at 25-30 for about 30-60 min. The reaction mixture temperature was raised to 35-40° C. and maintained for 1 hr. After completion of the reaction, in another flask 4000 ml DM water was charged and cooled to 10-15° C. and slowly added to the above reaction. The reaction mixture temperature was raised to 25-30° C. and maintained for 1 hr at same temperature; and the layers were separated. The aqueous layer was washed with 300 ml of dichloromethane and charged aqueous layer into RB flask and adjusted the pH 8.5-9.0 with 30% potassium carbonate solution. 800 ml of dichloromethane was charged and stirred for 15 min and separated layers. The aqueous layer was again extracted with 300 ml of dichloromethane. The organic layers were combined and washed with brine solution. The solvent was distilled out completely U/V at 35-40° C. and charged 350 ml of ethanol into the residue and the temperature was raised to 70-75° C. The reaction mixture was maintained for 30 minutes at 75-80° C. The reaction mixture was slowly cooled to 25-35° C. and stirred for 2-4 hrs. To the reaction mixture was charged 350 ml of methyl tertiary butyl ether, cooled to 0-5° C. and maintained 2 hrs at same temperature. The solid was filtered and washed with 100 ml tertiary butyl ether. The wet material was dissolved in 600 ml of methanol and the methanol was evaporated using spray drier at below 50° C. to obtain amorphous Linagliptin.

### Example 7

### Preparation of 1-[(4-methyl-quinazolin-2yl) methyl]-3-methyl-7-(2-butyn-1-yl)-8-[3-(R) amino-piperidin-1-yl]-xanthine (Linagliptin)

700 ml of Dichloromethane and 100 g of 1-[(4-methyl-quinazolin-2yl) methyl]-3-methyl-7-(2-butyn-1-yl)-8-[(R)-3-(tert.-butyloxycarbonylamino)-piperidin-1-yl]-xanthine (0.1746 moles prepared in example-4) were added into 1 lit. Round bottom flask equipped with overhead stirrer, thermo pocket and dropping funnel, at 25-30° C. The reaction mixture was washed with Sodium chloride solution and treated with activated charcoal followed by filtration through hyflo and washing with dichloromethane. The solvent was distilled out at 35-45° C. till ˜3.5 volumes dichloromethane remained inside. The reaction mixture was cooled to 25-35° C. and was slowly added 143 ml of trifluoroacetic acid for about 30-60 min. The reaction mixture temperature was raised to 35-40° C. and maintained for 1 hr. After completion of the reaction, 4000 ml of pre cooled DM water was added and cooled to 10-15° C., The reaction mixture the temperature was raised to 25-30° C. and maintained for 1 hr at same temperature. Layers were separated. The aqueous layer was washed with 300 ml of dichloromethane and the aqueous layer was taken into to RB flask and the pH was adjusted 8.5-9.0 with 30% potassium carbonate solution. 700 ml of dichloromethane was charged to the aqueous layer and stirred for 15 min; and layers were separated. The aqueous layer was again extracted with 300 ml of dichloromethane. The organic layers were combined and washed with brine solution. The solvent was distilled out completely under vacuum at 35-40° C. and was charged 350 ml of ethanol into the residue and the temperature was raised to 70-75° C. The reaction mixture was maintained for 30 minutes at 75-80° C., after that was slowly cooled to 25-35° C. and stirred for 2-4 hrs. To the reaction mixture was added 350 ml of methyl tertiary butyl ether and cooled to 0-5° C. and maintained 2 hrs at same temperature. The solid was filtered and washed with 100 ml tertiary butyl ether. The wet material was dissolved in 600 ml of methanol and the methanol was evaporated using spray drier at below 50° C.-80° C. to obtain amorphous Linagliptin.

We claim:

1. A process for the preparation of 8-[(3R)-3-aminopiperidin-1-yl]-7-(but-2-yn-1-yl)-3-methyl-1-[(4-methylquinazolin-2-yl)methyl]-3,7- dihydro-1H-purine-2,6-dione (Linagliptin) of compound of Formula I comprising the steps of:
a) purifying the protected compound of formula G, where in P is protecting group 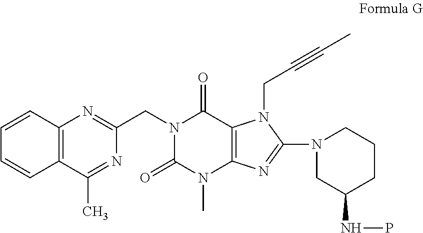
by treating with charcoal in a solvent, and
b) deprotecting the purified compound of formula G with an acid to give Linagliptin.

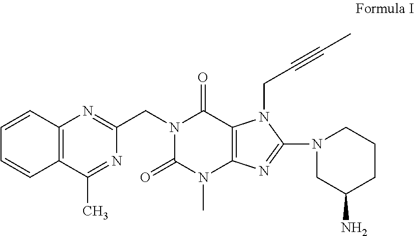 [SMILES: CC#CCn1c(N2CCC[C@@H](N)C2)nc2c1c(=O)n(Cc1nc(C)c3ccccc3n1)c(=O)n2C]

2. The process according to the claim 1, wherein the solvent is dichloromethane.

3. The process according to the claim 1, wherein the suitable acid is trifluoro acetic acid.

4. The process according to the claim 1, wherein protecting group P is di t-Butyl dicarbamate (Boc)

5. A process for the preparation of Linagliptin comprising the steps of:
a) brominating a methyl xanthine compound of formula A to give a bromo xanthine compound of formula B, 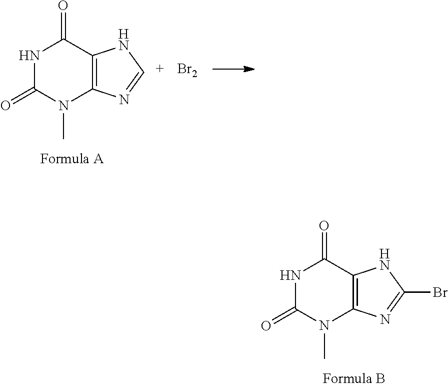
b) reacting the bromo xanthine compound of formula B with a butynyl compound of formula H to give a butyl xanthine compound of formula C, 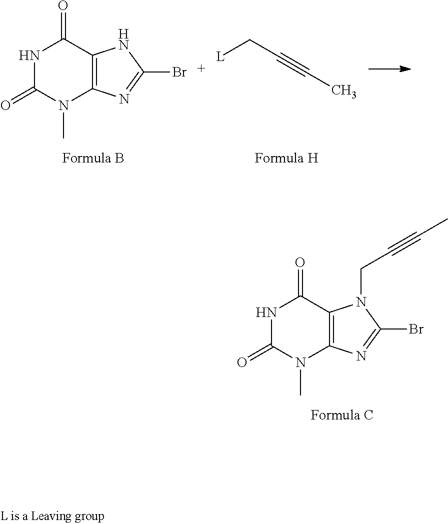
c) condensing the butyl xanthine compound of formula C with a quinazoline compound of formula D to give a compound formula E, 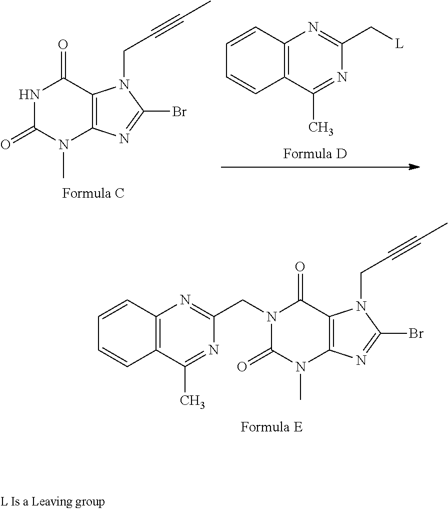
d) reacting the compound of formula E with a protected piperidine compound of formula F to give a protected Linagliptin compound of formula G, 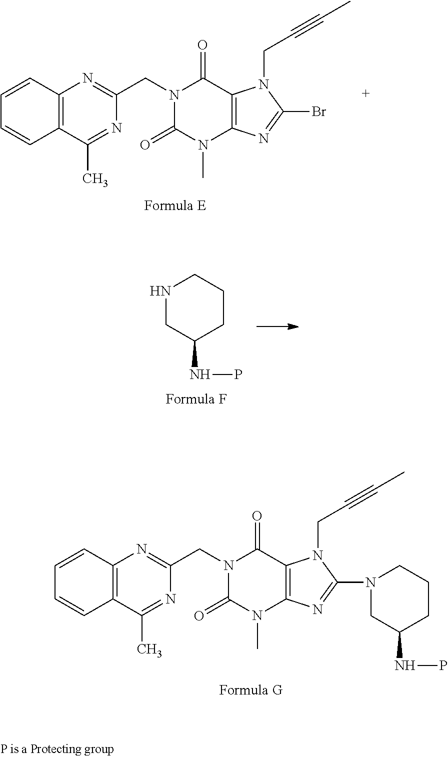
e) deprotecting the protected compound of formula G to give Linagliptin compound of formula I. 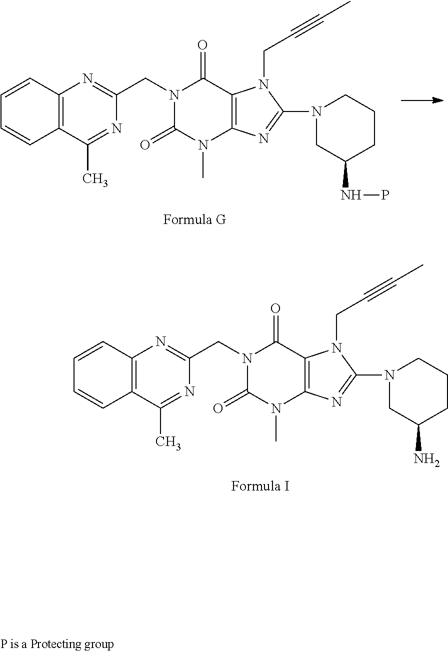
wherein, the products of stage a) to stage c) are purified by treating with an alcohol.

6. The process according to the claim 5, wherein bromination of a xanthine compound of formula A is carried out with liquid bromine in presence of a metal acetate such as sodium acetate and a suitable solvent.

7. The process according to the claim 5, wherein reaction of the bromoxanthine compound of formula B with a butynyl compound of formula H is carried out in presence of a base and solvent.

8. The process according to the claim 5, wherein the condensation of the butyl xanthine compound of formula C, with a quinazoline compound of formula D is carried put in the presence of a base, suitable solvent and optionally in presence of a phase transfer catalyst.

9. The process according to the claim 5, wherein the reaction of the compound of formula E with a protected piperidine compound of formula F is carried out in presence of a base, solvent and optionally in presence of a metal halide.

10. The process according to the claim 5, wherein the deprotection is carried out by treating the protected compound of formula G with an acid.

11. The compound of Formula G, which is characterized by PXRD as depicted in FIG. 1. 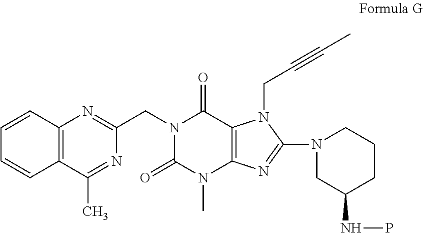
wherein P is tert.butoxy carbonyl.

12. The use of compound of claim 11, in the preparation of Linagliptin.
